(19) United States
(12) Patent Application Publication
(10) Pub. No.: US 2015/0151095 A1
(43) Pub. Date: Jun. 4, 2015

(54) COMBINED BALLOON DILATION CATHETER AND INTRODUCER FOR DILATION OF EUSTACHIAN TUBE

(71) Applicant: MUAAZ TARABICHI, DUBAI (AE)

(72) Inventor: MUAAZ TARABICHI, DUBAI (AE)

(21) Appl. No.: 14/460,039

(22) Filed: Aug. 14, 2014

Related U.S. Application Data
(60) Provisional application No. 61/910,151, filed on Nov. 29, 2013.

(51) Int. Cl. A61M 29/02

(52) U.S. Cl. CPC A61M 29/02; A61M 2029/025; A61M 2210/0675

(57) ABSTRACT

A Eustachian tube dilation device adapted for insertion via an ear canal and including a balloon catheter having a sheath and a balloon mounted proximate to a distal end of the sheath; and an introducer having a malleable distal portion and a more rigid proximal portion. The introducer defines a lumen that is capable of slidably receiving the balloon catheter within the introducer and has a distal opening through which the balloon can be extended from the introducer into the Eustachian tube. The Eustachian tube dilation device may further comprise an inflation device. The Eustachian tube dilation device may also incorporate imaging technology and endoscopy. A method of using the Eustachian tube dilation device for dilation of the Eustachian tube via the ear canal is also described.

This application claims the benefit of U.S. Provisional Application Ser. No. 61/910,151 filed Nov. 29, 2013, the content of which are hereby incorporated by reference.

### BACKGROUND OF THE INVENTION

1. Field of the Invention

The present invention is directed to balloon catheters suitable for dilating Eustachian tubes. In particular, the present invention is directed to a Eustachian tube dilation device including a balloon catheter and a malleable introducer.

2. Description of the Related Technology

Eustachian tube is a narrow, one-and-a-half inch long channel connecting the middle ear with the nasopharynx in back of the nose. The Eustachian tube functions as a pressure equalizing valve for the middle ear which is normally filled with air. When functioning properly, the Eustachian tube opens for a fraction of a second periodically (about once every three minutes) in response to swallowing or yawning. In so doing, it allows air into the middle ear to replace air that may have been absorbed by the middle ear lining (mucous membrane) or to equalize pressure changes occurring on altitude changes.

Obstruction or blockage of the Eustachian tube results in a negative middle ear pressure, with retraction (sucking in) of the eardrum. In adults, this is usually accompanied by some ear discomfort, a fullness or pressured feeling and may result in a mild hearing impairment and head noise (tinnitus). There may be no symptoms in children. If the obstruction is prolonged, fluid may be drawn from the mucous membrane of the middle ear, creating a condition referred to as serous otitis media (fluid in the middle ear). This occurs frequently in children in connection with an upper respiratory infection and accounts for the hearing impairment associated with this condition.

When the Eustachian tube contains a build-up of fluid, a number of conditions will occur. First, the body absorbs air from the middle ear, causing a vacuum to form which tends to pull the lining membrane and ear drum inward, causing pain. Next, the body replaces the vacuum with more fluid which tends to relieve the pain, but the patient can experience a fullness sensation in the ear. Treatment of this condition with antihistamines and decongestants can take many weeks to be fully effective. Finally, the fluid can become infected, which is painful and makes the patient feel ill and causes hearing loss. If the inner ear is infected, the patient may feel a spinning or turning sensation (vertigo). The infection is typically treated with antibiotics.

Recently, several devices have been developed for dilating the distal (nose side) of the Eustachian tube. Such devices include balloon catheters to be introduced through the nose and into the Eustachian tube. Once inserted, the balloon is inflated to dilate the distal part of the Eustachian tube.

US 2009/0076446 discloses a balloon catheter for insertion into a nasal passageway. The balloon catheter includes a first tubular segment, which has multiple zones of differing deformability along its length. The catheter further includes a coaxially aligned hypotube, which is positioned within the lumen of the first tubular segment and extends from proximate to the distal end of the first tubular segment. The hypotube may run the length of the first tubular segment, or may run along only a portion of the length of the first tubular segment. The hypotube terminates with an atraumatic tip and includes a balloon dilator affixed near the tip. An aperture near the tip of the hypotube fluidly connects the interior of the balloon with the lumen of the hypotube enabling the balloon to selectively expand and contract. The first tubular segment may be shaped for adapting to the anatomy of the nasal passageway.

US 2010/0198191 discloses a device for accessing a Eustachian tube through the nasal passageway. The device includes an elongate rigid sheath, which may be deformed to adapt to the geometry of the nasal passageway. The device also has an elongated flexible insert coupled to the distal portion of the rigid sheath. The insert preferentially has a lateral stiffness such that when inserted into a Eustachian tube, the insert will conform to the pathway of the Eustachian tube and not cause significant deformation of the Eustachian tube. The insert may also include a preformed shape adapted for the anatomy of the Eustachian tube. The insert may include a core wire and an expandable balloon. The balloon may have a preformed shape which matches the profile of a Eustachian tube. The device may further include an atraumatic tip in the shape of a ball and a lumen for passage of fluids. The device does not require a guidewire for insertion into a Eustachian tube.

US 2011/0264134 discloses a balloon dilation catheter that includes a substantially rigid inner guide member and a movable sheath coupled to a balloon that is slidably mounted on the substantially rigid inner guide member. The balloon dilation catheter is introduced into a sinus cavity of a subject using a substantially rigid inner guide member via a nasal passageway. The sheath and balloon are advanced in a distal direction over the substantially rigid inner guide member to place the balloon in the drainage pathway. The balloon is inflated to expand or otherwise remodel the drainage pathway. The device relies on the substantially rigid inner guide member in the sheath to introduce the balloon.

Examining the proximal part of the Eustachian tube (the ear side) is possible through a perforated tympanic membrane or when surgery is performed for a condition called cholesteatoma that results in disruption of the tympanic membrane. However, this examination is not common practice because it cannot be done by microscope, an essential instrument in ear surgery. Visualization of the Eustachian tube from the ear side requires an angled endoscope, because there is an angle of 30 degrees or more between the axis of the Eustachian tube and the ear canal. In addition, the medial wall of the Eustachian tube has the carotid artery and attempted manipulation of that area is limited by concerns about the integrity of carotid artery especially when doing this blindly. Intubation with a stiff catheter without guiding it to the right opening passageway may result in cannulation of the carotid artery, pain, discomfort, injury to the carotid artery, stroke and death.

The present invention provides a Eustachian tube dilation device suitable for accessing the Eustachian tube through ear canal. The Eustachian tube dilation device is capable of maneuvering through the passageway from the ear canal to the Eustachian tube, and avoiding obstructions and the carotid artery and causes minimal discomfort for patients.

### SUMMARY OF THE INVENTION

In one aspect, the present invention provides a Eustachian tube dilation device comprising a balloon catheter having a sheath and a balloon mounted proximate to the distal end of the sheath. The device also includes an introducer having a malleable distal portion and a more rigid proximal portion. The introducer includes a lumen that is capable of slidably receiving the balloon catheter within the introducer.

In another aspect, the Eustachian tube dilation device of the present invention further comprises an inflation device.

In yet another aspect, the Eustachian tube dilation device of the present invention comprises a sheath with an aspiration lumen.

In yet another aspect, the Eustachian tube dilation device of the present invention further comprises an adapter device.

In yet another aspect, the Eustachian tube dilation device of the present invention comprises an introducer with a malleable portion comprising a plurality of zones of differing malleability.

In yet another aspect, the present invention provides a method of dilating Eustachian tube of a subject with the Eustachian tube dilation device of the present invention. In the method, a passageway from the ear canal to the Eustachian tube of the subject is inspected to determine the geometry of the passageway; the introducer is deformed to a configuration substantially matching the geometry of the passageway and then inserted through the passageway towards the Eustachian tube. Subsequently, the balloon catheter is inserted through the introducer to place the balloon in the opening of the Eustachian tube; and the balloon is inflated to dilate the Eustachian tube.

In yet another aspect, the present invention provides a method of dilating a Eustachian tube of a subject with the Eustachian tube dilation device of the present invention. The method includes inspecting a passageway from the ear canal to the Eustachian tube of the subject to determine the geometry of the passageway; shaping the introducer to a configuration substantially matching the geometry of the passageway; inserting the balloon catheter into the shaped introducer; inserting the shaped introducer with the balloon catheter through the passageway towards the Eustachian tube; adjusting the position of the balloon to place the balloon in the opening of the Eustachian tube; and inflating the balloon to dilate the Eustachian tube.

In yet another aspect, the method of the present invention comprises the step of lubricating the exterior surface of the introducer with an anesthetizing lubricant.

In still yet another aspect, the present invention may incorporate an endoscope integrated with the introducer or the balloon that confirms visually the location of the device in relationship to the carotid artery.

In another aspect, the introducer or the balloon can incorporate a sensor coil that is wired to an image navigation system that allows the determination of the location of the tip of the introducer or the catheter in relationship to the patient anatomy.

### BRIEF DESCRIPTION OF THE DRAWINGS

FIG. 1A is a diagram showing a balloon catheter with a sheath and a balloon located proximate to the distal end of the sheath, according to one embodiment of the present invention.

FIG. 1A is a diagram showing an introducer according to one embodiment of the present invention.

FIG. 1C is a diagram showing a Eustachian tube dilation device including both the balloon catheter and introducer, as assembled according to one embodiment of the present invention.

FIG. 2A is a diagram showing the balloon catheter with an inflation lumen that opens to the interior of the balloon, according to one embodiment of the present invention.

FIG. 2B is a diagram showing the balloon catheter with both an inflation lumen and aspiration lumen, according to one embodiment of the present invention.

FIG. 3 is a diagram showing an introducer with fins, according to one embodiment of the present invention

FIG. 4 is a flow chart showing a method of dilating the Eustachian tube via the ear canal using an embodiment of a Eustachian tube dilation device, according to one embodiment of the present invention.

FIG. 5 is a diagram showing an embodiment of Eustachian tube dilation device of the present invention inserted through ear canal to reach and dilate the Eustachian tube.

### DETAILED DESCRIPTION OF THE PREFERRED EMBODIMENT(S)

For illustrative purposes, the principles of the present disclosure are described by referencing various exemplary embodiments. Although certain embodiments are specifically described herein, one of ordinary skill in the art will readily recognize that the same principles are equally applicable to, and can be employed in other devices and methods. Before explaining the disclosed embodiments of the present disclosure in detail, it is to be understood that the disclosure is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

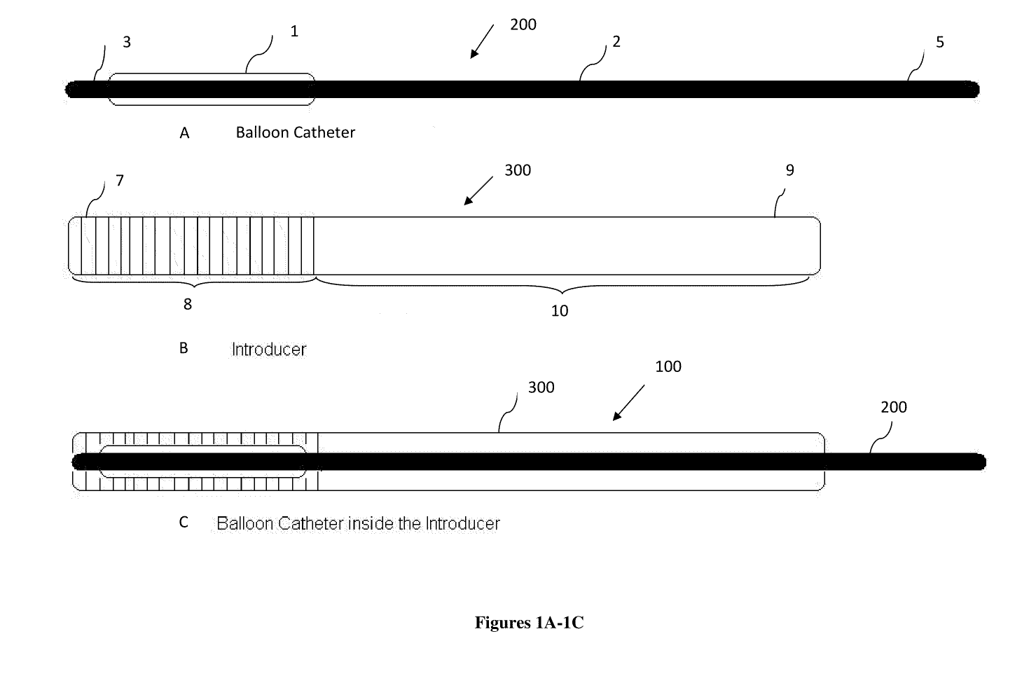

The present invention provides in a first embodiment a Eustachian tube dilation device 100 comprising a balloon catheter 200 and an introducer 300 as shown in FIG. 1A. The balloon catheter 200 has a sheath 2 with a distal end 3 and proximal end 5. The balloon catheter 200 may have a length that is shorter than a typical balloon catheter that is employed for insertion through the nasal passageway to the Eustachian tube since the balloon catheter 200 of the present invention is designed for insertion via the ear to the Eustachian tube. A balloon 1 is mounted at or near the distal end 3 of the sheath 2.

Referring to FIG. 1B, introducer 300 may be in the form of a hollow tube that defines a lumen. Introducer 300 has a distal portion 7 and a proximal portion 9. There is a malleable portion 8 located in the distal portion 7 of the introducer 300. Malleable portion 8 is deformable so that it can be deformed to a variety of different potentially desirable shapes for customization of introducer 300 to a particular patient's ear cavity in use. The remaining portion 10 of the introducer 300 may be relatively rigid as compared to malleable portion 8 or portion 10 may be rigid. The balloon catheter 200 is configured to slidably fit inside of the introducer 300, to form the Eustachian tube dilation device 100 shown in FIG. 1C.

The passageway from the ear canal to the Eustachian tube refers to the ear canal, passing through the disrupted tympanic membrane, the middle ear and the Eustachian tube. This passageway typically has an angle between ear canal and Eustachian tube at from about 20° to about 50° or from about 25° to about 40° as measured between the ear canal and the Eustachian tube.

As used herein, the distal end is the end of the device that designed to be inserted proximate to the Eustachian tube, while the proximal end of the device is the end of the device intended to remain outside of the passageway from the ear canal to the Eustachian tube.

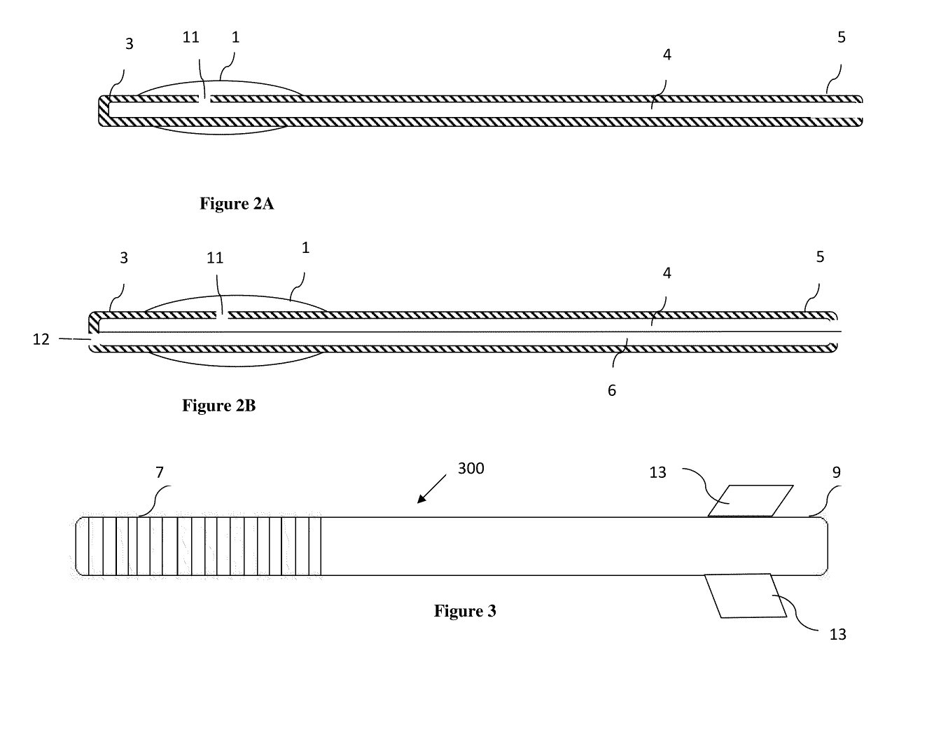

Referring to FIG. 2A, the balloon catheter 200 comprises a sheath 2, which may be in the form of a cylindrical tube that defines an inflation lumen 4 with an opening in the proximal end 5. Within the sheath 2 is an inflation lumen 4 extending substantially the entire length of the sheath 2 from the distal end 3 to the proximal end 5. The inflation lumen 4 has an inflation aperture 11 at or near the distal end 3 of the sheath 2, where the inflation aperture 11 fluidly communicates with the interior of the balloon 1. The balloon 1 may be inflated by providing a fluid to the balloon 1 through the inflation lumen 4 within the sheath 2. The balloon 1 may be deflated by removing the fluid from balloon 2 via inflation lumen 4. The fluid used for inflation of the balloon 1 may be water, saline, nitrogen, other gas or a gas mixture (such as air).

Sheath 2 may be a structure which has high bending elastic modulus as well as a certain degree of flexibility. There are many materials that may be used for fabricating the sheath 2. Non-limiting categories of suitable sheath materials include metals, synthetic resins and composite materials including resins strengthened by metal. Examples of suitable metals include stainless steel, tungsten, nickel-titanium alloy. The synthetic resins may comprise any polymeric material that is approved for human use and which is sufficiently rigid for fabricating the sheath 2. The polymer material may be selected from polyolefins such as polyethylene and polypropylene; polyamides such as nylons; polyesters, polyurethanes, polyethylene terephthalate and polybutylene terephthalate; and fluorine-based resins. In some embodiments, the polymer material may be polyisoprene, latex rubber, and/or polyvinyl chloride.

The balloon 1 is mounted proximate to the distal end 3 of the sheath 2. In some embodiments, the balloon is about 1.5-2.0 cm in length as measured along the longitudinal axis of the device.

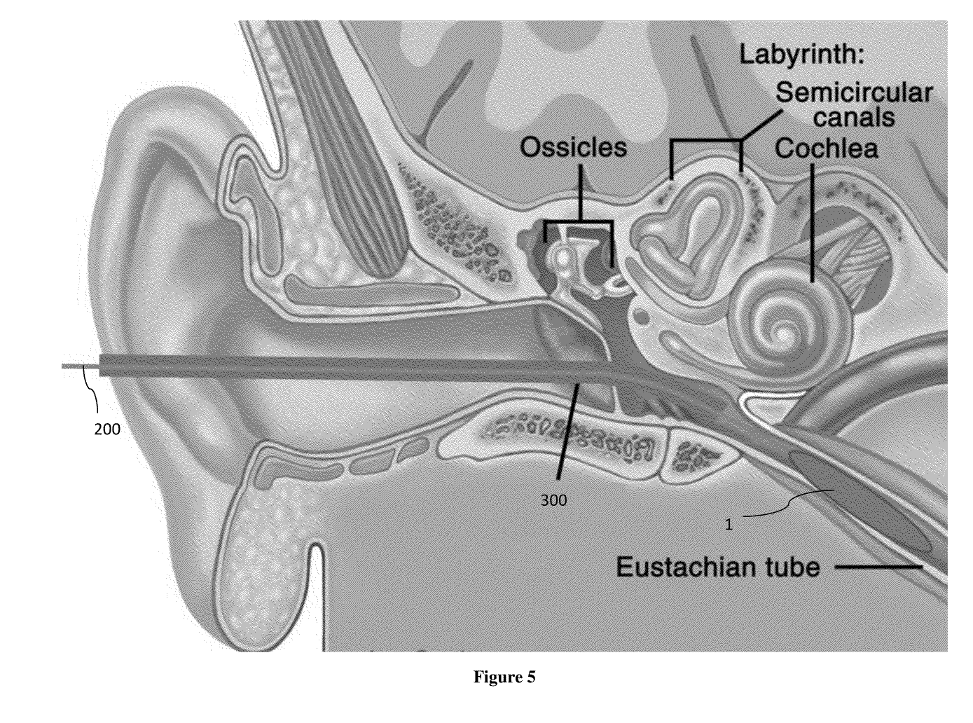

A portion of the distal end 3 of sheath 2 may be provided with a coating on the exterior surface to aid bonding with the balloon 1. Once the balloon is properly positioned in the Eustachian tube, a fluid is provided to the balloon 1 through the inflation aperture 11 from inflation lumen 4 within the sheath 2. Inflation of the balloon 1 to a fully inflated state causes the Eustachian tube to dilate as shown in FIG. 5. The balloon 1 may be inflated only a single time or, alternatively, the balloon 1 may be inflated, deflated, and inflated again a plurality of times in order to achieve the desired degree of dilation of the Eustachian tube. Optionally, each inflation step may be performed after positioning the balloon 1 in a different position within the Eustachian tube.

The wall of the balloon 1 has sufficient strength to withstand relatively high inflation pressures of from about 6 atmospheres to about 20 atmospheres, or from about 8 atmospheres to about 18 atmospheres, or from about 10 atmospheres to about 16 atmospheres, or from about 12 atmospheres to about 14 atmospheres.

The material for fabricating the balloon 1 may be a high strength, flexible polymeric material including urethane based elastomers, copolymers of polyurethane and silicon, polyamides (e.g., Nylon), PEBAX® and the like. Examples of methods of forming the balloon 1 include dip-forming and blow molding.

The balloon 1 is in a deflated form during insertion and positioning of balloon catheter 200 in the opening of the Eustachian tube from the ear canal side. Once the balloon 1 is properly positioned in the Eustachian tube, it may then be inflated using any suitable inflation device (not shown) that is coupled to the proximal end 5 of the sheath 2. One exemplary inflation device that may be used is described in US 2010/0211007, which is incorporated herein by reference in its entirety. Of course, many other inflation devices known to a person skilled in the art may also be used, such as a syringe or the like.

The inflated balloon 1 may be deflated and withdrawn from the Eustachian tube. Deflation of the balloon 1 is accomplished by reducing the fluid pressure within the interior of the balloon 1. For example, the plunger of a syringe or the like that is fluidly coupled to inflation lumen 4 at the proximal end 5 of the sheath 2 may be used to remove fluid from the interior of the balloon 1. The balloon catheter 200 can then be withdrawn from the Eustachian tube by pulling in the proximal direction.

Referring to FIG. 2B, in some embodiments, the sheath 2 optionally has an aspiration lumen 6 extending from the distal end 3 to the proximal end 5. The aspiration lumen 6 has an aspiration aperture 12 at the or near the distal end 3 of the sheath 2. The aspiration lumen 6 may be used to retrieve fluid from the Eustachian tube or middle ear by aspiration via the aspiration aperture 12.

In some embodiments, the proximal end 5 of the sheath 2 may be attached to an adapter device. The adapter device typically includes inlets and passageways through which the inflation lumen 4 and, optionally the aspiration lumen 6, of the sheath 2 may be accessed. The adapter device may also include a flange surrounding the inlets. The adapter device may alternatively comprise a conventional hub or manifold device. For example, an inflation device may be attached to the adapter device and be used to inflate and deflate the balloon 1 on the distal end 3 of the sheath 2 via the inflation lumen 4. Several embodiments of a suitable adapter device are described in, for example, US 2009/0076446, which is incorporated herein by reference in its entirety.

The sheath 2 has a length in the range of from about 4 cm to about 10 cm, or from about 5 cm to about 9 cm, or from about 5 cm to about 8 cm, or from about 6 cm to about 8 cm. In some embodiments, the sheath 2 may comprise a soft plastic grip at the proximal end 5 to assist the user in manipulating the balloon catheter 200.

In some embodiments, the exterior surface of the sheath 2 has markers that represent the distance from the tip at the distal end 3 of the sheath 2. The markers can assist a user in estimation of the location of the balloon 2 in the ear canal or Eustachian tube.

The introducer 300 has a tubular structure with a length slightly shorter than the balloon catheter 200. The length of the introducer 300 may be from about 3 cm to about 9 cm, or from about 4 cm to about 8 cm, or from about 4 cm to about 7 cm, or from about 5 cm to about 7 cm.

The introducer 300 is divided into at least two portions, a malleable portion 8 and a portion 10 that is more rigid than the malleable portion 8. The malleable portion 8 is located at the distal portion 7 of the introducer 300 and may be deformed or shaped by a user to form a desired shape or configuration. For example, the malleable portion 8 may be shaped to form an angle of from greater than 0° to 60°, or from about 10° to about 50°, or from about 20° to about 40°. In operation, the malleable portion 8 may be curved to form a shape configured to match the contour and geometry of the passageway from the ear canal to the Eustachian tube. The length of the malleable portion 8 may be from about 20% to about 50% of the introducer 300, or from about 25% to about 40% of the introducer 300, or from about 28% to about 38% of the introducer 300, or from about 30% to about 36% of the introducer 300, or from about 32% to about 35% of the introducer 300.

The malleable portion 8 of the introducer 300 may be made of any malleable material such as a plastic, metal or a combination thereof, having physical properties that allow the malleable portion 8 to be shaped or deformed by hand and substantially retain its shape. Some malleable metals suitable for fabricating the malleable portion 8 include annealed copper (with coating or treatment to make it biocompatible), anodized annealed aluminum, annealed stainless steel, nitinol, and brass.

In one embodiment, the malleable portion 8 comprises annealed stainless steel tubing having a carbon content sufficient to make it hand shapeable by the user. In addition, the annealed stainless steel tubing should also have a carbon content sufficient to provide adequate stiffness to substantially maintain the preset shape when navigating through the passageway from the ear canal to the Eustachian tube.

In some embodiments, introducer 300 is fabricated from aluminum that is malleable such as “dead soft” types of aluminum (e.g., annealed 3003 aluminum as known to a person skilled in the art). As used herein, the term “dead soft” means that a metal is capable of being deformed to a particular shape and of holding that shape until it is reformed to a new shape or back to its original shape.

Some metals, such as copper, may require a coating of plastic about any exposed surface so as to prevent any reaction by the metal with its surrounding environment. Such a plastic coating allows a metal such as copper to maintain a pristine state while providing an inert contact surface for any materials passing through or about the introducer 300.

The malleable portion 8 may also be made from a malleable plastic. The malleable plastic materials suitable for fabricating the malleable portion 8 include, polyesters (e.g., polyethylene terephthalate), polyethylenes (e.g., polytetrafluoroethylene), polypropylenes, perfluoroalkoxy (e.g., Teflon-PFA® sold by DuPont), and copolymers, derivatives thereof.

In some embodiments, malleable portion 8 of the introducer 300 may have non-uniform malleability along its length. The malleable portion 8 may, for example, be comprised of multiple zones of differing malleability or the malleability of malleable portion 8 may vary gradually along its length. By enabling the malleability of the malleable portion 8 to vary along its length, the desired strength and flexibility characteristics of the introducer 300 can be optimized for the anatomy of the passageway from the ear canal to the Eustachian tube.

For example, the malleable portion 8 may be designed to include a first zone a the proximal end of the malleable portion 8 that is relatively stiff and has a relatively low malleability to improve its ability to transmit longitudinal forces; a second intermediate zone having a relatively higher malleability which is optimized to transmit rotational displacement while maintaining its cross sectional profile; and a third zone that is more flexible and having a relatively high malleability to improve its ability to travel through intricate passageway from the ear canal to the Eustachian tube. The length of the various zones of malleability may vary. Moreover, each zone may be designed so that the malleability gradually increases or decreases within the particular zone.

The malleable portion 8 of the introducer 300 may have a hoop strength sufficient to prevent the introducer 300 from flattening when the malleable portion 8 is shaped. For example, the malleable portion 8 can be made of stainless steel or any other suitable material that will allow for the flexibility of the malleable portion 8 while at the same time preventing flattening by having a sufficient hoop strength. This prevents the malleable portion 8 from kinking or flattening when shaped. As a result, the introducer 300 may be shaped into various configurations without kinking or flattening and it can stay in its shaped configuration until a user straightens or rebends the introducer 300.

The portion 10 of the introducer 300 that is relatively rigid is located at the proximal portion 9 of the introducer 300, which accounts for the remaining length of the introducer 300 other than the malleable portion 8. Portion 10 is not malleable and has sufficient strength for providing support to the malleable portion 8 for inserting the introducer 300 into the passageway from the ear canal to the Eustachian tube. There are many medically approved rigid materials that may be used for fabricating the portion 10 of the introducer 300. Some suitable materials include synthetic resins such as polyethylene, polypropylene, polyethylene terephthalate, polyamide, polyvinyl chloride, polyurethane and fluorine contained resins.

The introducer 300 defines a lumen that can slidably receive the balloon catheter 200 within the interior lumen of introducer 300 as shown in FIG. 1C. The inner diameter of the lumen of introducer 300 may be from about 0.8 mm to about 1.5 mm, or from about 0.9 mm to about 1.4 mm, or from about 1.0 mm to about 1.4 mm, or from about 1.1 mm to about 1.3 mm. The outer diameter of the introducer 300 may be from about 1.3 mm to about 2.3 mm, or from about 1.4 mm to about 2.2 mm, or from about 1.5 mm to about 2.1 mm, or from about 1.6 mm to about 2.0 mm, or from about 1.7 mm to about 1.9. The thickness of the wall of the introducer 300 may be from about 0.3 mm to about 0.8 mm, or from about 0.3 mm to about 0.7 mm, or from about 0.4 mm to about 0.6 mm. The thickness of the wall should be sufficient to ensure suitable strength for navigating the introducer 300 through the passageway from the ear canal to the Eustachian tube.

The exterior surface of the introducer 300 may be coated with a hydrophilic polymer material such as polyethylene oxide to facilitate insertion and removal of the introducer 300 from the passageway from the ear canal to the Eustachian tube. In addition, the interior surface of the introducer 300 may be coated with a lubricious or low friction coating to reduce frictional contact forces when sliding the balloon catheter 200 within the introducer 300.

In some embodiments, the distal portion 7 of the introducer 300 has a tapered shape, such that the distal portion 7 becomes narrower in the direction approaching the distal end of the device, so as to facilitate the insertion of the introducer 300 through the passageway from ear canal to Eustachian tube. In one embodiment, in this tapered shape, an outer diameter of the distal end of the introducer 300 is smaller by about 0.05 mm to about 0.20 mm than an outer diameter of another portion of the distal portion 7 of the introducer 300.

In some embodiments, the proximal portion 9 of the introducer 300 may include fins 13 to enable a user to better manipulate the introducer 300 in the passageway from ear canal to Eustachian tube as shown in FIG. 3. More than one pair of fins 13 may be used. The fins 13 do not have to be positioned directly opposite each other, and configurations of odd-numbered fins 13 may also be used.

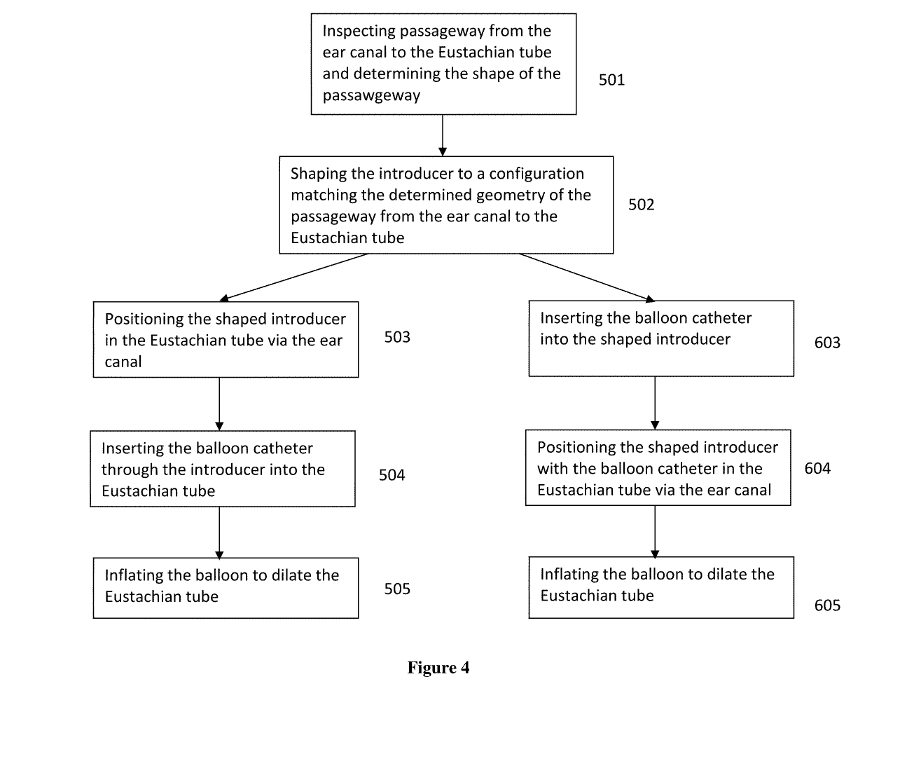

In another aspect, the present invention provides a method of using the using the Eustachian tube dilation device 100 of the present invention for dilating the Eustachian tube. FIG. 4 illustrates one embodiment of the method of dilating the Eustachian tube. Before shaping the malleable portion 8 of the intruder 300 to match the geometry of the passageway from ear canal to Eustachian tube, the passageway needs to be visually inspected. In some embodiments, inspecting 501 the passageway from ear canal to Eustachian tube of a patient may be carried out by an imaging device. The term “imaging device” is defined as including fiberscopes, endoscopes and all other long, thin, flexible viewing instruments used by surgeons and those skilled in the art to view the interior of body cavities.

Based on the visual inspection of the passageway from ear canal to Eustachian tube, the contour and geometry of the passageway is determined. The next step of the method is shaping 502 the malleable portion 8 of the introducer 300 to a configuration matching the geometry of the passageway from the ear canal to the Eustachian tube. The user is able to deform the malleable portion 8 into a shape that substantially matches the determined configuration of the passageway from the ear canal to the Eustachian tube. The user may accomplish this by hand by deforming the malleable portion 8 of the introducer 300 into almost any configuration. The shaped introducer 300 is thus adapted to maneuver through the bends, curves, and other articulations present within the passageway from ear canal to Eustachian tube of a particular patient with as little obstruction as possible.

In some embodiments, the next step of the method is positioning 503 the shaped introducer 300 through the passageway from the ear canal to the Eustachian tube towards the opening of the Eustachian tube. The insertion of shaped introducer 300 may be guided by an imaging device. In one embodiment, during the insertion step, a user may find that the shape of the shaped introducer 300 is not optimal for fitting the geometry of the passageway from the ear canal to the Eustachian tube, e.g. as a result of encountering an obstruction during insertion. The user may then retract the shaped introducer 300 and further alter the shape of the malleable portion 8 of the introducer 300 to ensure that its configuration better matches the geometry of the passageway. The reshaped introducer 300 may then be reinserted into the passageway via the ear canal. The exterior surface of the introducer 300 may be treated with an anesthetizing agent or a lubricant (such as Lubafax™ jelly). The shaped introducer 300 may reach the Eustachian tube via the ear canal without trauma and avoiding causing stress to the patient.

After the shaped introducer 300 is properly positioned, the next step is inserting 504 the balloon catheter 200 through the properly positioned introducer 300 to locate the balloon 1 in the Eustachian tube. The user can verify the placement of the balloon 1 using an imaging device.

Inflating 505 the balloon 1 may then be carried out by introducing a fluid, such as water, saline or air through the inflation lumen 4 into the balloon 1. The fluid will inflate the balloon 1, thus dilating the Eustachian tube as shown in FIG. 5. The balloon 1 may be inflated only a single time or, alternatively, the balloon 1 may be inflated, deflated, and re-inflated one or more times in order to achieve the desired degree of dilation or widening of the Eustachian tube. Each inflation step 505 may be performed with the balloon 1 in the same or a different position, e.g. after repositioning the balloon 1 in a different position within the Eustachian tube. The balloon 1 is then deflated and withdrawn into the shaped introducer 300 and the shaped introducer 300 is withdrawn in a proximal direction from the ear canal. The dilated Eustachian tube may optionally be inspected after the withdrawal of the balloon catheter 200 to verify the successful completion of the procedure.

Referring to FIG. 4, an alternative embodiment of the method of the present invention is also shown. In this alternative embodiment, the first two steps: inspecting 501 the passageway from ear canal to Eustachian tube and shaping 502 the introducer to a configuration matching the geometry of the passageway from the ear canal to Eustachian tube are the same as described above. The next step in this alternative embodiment is inserting 603 the balloon catheter 200 into the shaped introducer 300, before the shaped introducer 300 is inserted into the passageway from the ear canal to the Eustachian tube. It is preferred that the tip at distal end 3 of the balloon catheter 200 does not extend beyond the distal end of the introducer 300 FIG. 1C during positioning of introducer 300 in the Eustachian tube via the ear canal as shown in FIG. 1C.

The next step in this alternative embodiment is positioning 604 the shaped introducer 300 with the balloon catheter 200 inside into the passageway from the ear canal to the Eustachian tube. In this embodiment, the balloon catheter 200 and the shaped introducer 300 are positioned together as a single unit. Positioning of the shaped introducer 300 and balloon catheter 200 may be guided and/or verified by use of an imaging device.

After the shaped introducer 300 is properly positioned, the next step is adjusting 505 the position of the balloon catheter 200 to place the balloon 1 into the Eustachian tube. The user can verify the placement of the balloon 1 using the imaging device. The properly placed balloon 1 is then inflated by introducing a fluid, such as water, saline or air through the inflation lumen 4 into the balloon 1. The fluid will inflate the balloon 1, thus dilating the Eustachian tube as shown in FIG. 5.

The balloon 1 may be inflated only a single time or, alternatively, the balloon 1 may be inflated, deflated, and re-inflated one or more times in order to achieve the desired degree of dilation or widening of the Eustachian tube. Each inflation step may be performed at the same or different location of the balloon 1 as discussed above. The balloon 1 is then deflated and repositioned within the shaped introducer 300 and the shaped introducer 300 is withdrawn in a proximal direction from the ear canal. The dilated Eustachian tube may then be inspected after withdrawal of the introducer 300 to verify successful completion of the procedure.

In some embodiments, when the sheath 2 of the balloon catheter 200 has an aspiration lumen 6 and an aspiration aperture 12, the method may further comprise a step of aspirating a fluid from any location in the passageway from the ear canal to the Eustachian tube, especial at the middle ear or Eustachian tube. This step may be very beneficial when there is a fluid accumulated or trapped in the middle ear or in the Eustachian tube. Aspiration of fluid may be done before inspecting the passageway to enhance visualization thereof.

The Eustachian tube dilation device 100 enables a user to place and inflate a balloon in the opening of Eustachian tube through the passageway from the ear canal to the Eustachian tube without intubating the other blind pouches that usually exist and without damaging the carotid artery or other middle ear structures. The introducer 300 may be shaped to a configuration that directs the introducer 300 to clear the carotid and other blind pouches in the passageway.

With respect to the ear there are multiple false passages that can be cannulated. Because there are many false passages, in some embodiments of the present invention imaging technology is used in order to guide a catheter into the right orifice and to avoid potential physical harm, such as damage to the carotid artery. The present invention may incorporate an endoscope integrated with the introducer or the balloon that confirms visually the location of the device in relationship to the carotid artery. It is additionally contemplated that the introducer or the balloon can incorporate a sensor coil that is wired to an image navigation system that allows the determination of the location of the tip of the introducer or the catheter in relationship to the patient anatomy.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A Eustachian tube dilation device adapted for insertion via an ear canal, said device comprising:
a balloon catheter having a sheath defining an inflation lumen and a balloon mounted proximate to a distal end of the sheath; and
an introducer having a malleable distal portion and a proximal portion, the introducer defining a lumen configured for slidably receiving the balloon catheter within the introducer and having a distal opening through which the balloon catheter can be extended outside of the introducer.

2. The Eustachian tube dilation device of claim 1, wherein the sheath comprises an inflation lumen extending to a proximal end of the balloon catheter and the inflation lumen has an inflation aperture fluidly communicating with an interior of the balloon.

3. The Eustachian tube dilation device of claim 1, wherein the balloon comprising a material selected from the group consisting of urethane based elastomer, copolymer of polyurethane and silicon, polyamides and combinations thereof.

4. The Eustachian tube dilation device of claim 1, further comprising an inflation device.

5. The Eustachian tube dilation device of claim 1, wherein the sheath has a length of from about 4 cm to about 10 cm, or from about 5 cm to about 9 cm, or from about 5 cm to about 8 cm, or from about 6 cm to about 8 cm.

6. The Eustachian tube dilation device of claim 1, wherein the sheath further comprises a plurality markers on the exterior surface for representing a distance from the distal end of the sheath.

7. The Eustachian tube dilation device of claim 1, wherein the introducer has a length of from about 3 cm to about 9 cm, or from about 4 cm to about 8 cm, or from about 4 cm to about 7 cm, or from about 5 cm to about 7 cm.

8. The Eustachian tube dilation device of claim 1, wherein the malleable portion comprises from about 20% to about 50% of a length of the introducer, or from about 25% to about 40% of the length of the introducer, or from about 28% to about 38% of the length of the introducer, or from about 30% to about 36% of the length introducer, or from about 32% to about 35% of the length of the introducer.

9. The Eustachian tube dilation device of claim 1, wherein the malleable portion comprises a material selected from the group consisting of a metal, a plastic and a combination thereof.

10. The Eustachian tube dilation device of claim 9, wherein the metal is selected from the group consisting of annealed copper, anodized annealed aluminum, annealed stainless steel, nitinol, and brass.

11. The Eustachian tube dilation device of claim 9, wherein the plastic is selected from the group consisting of polyesters, polyethylenes, polypropylenes, and fluorinated polymers.

12. The Eustachian tube dilation device of claim 1, wherein the malleable portion comprises a plurality zones of differing malleability.

13. The Eustachian tube dilation device of claim 1, wherein the introducer has a wall thickness of from about 0.3 mm to about 0.8 mm, or from about 0.3 mm to about 0.7 mm, or from about 0.4 mm to about 0.6.

14. The Eustachian tube dilation device of claim 1, wherein the introducer comprises at least one fin on an exterior surface of a proximal portion of the introducer.

15. A method of dilating a Eustachian tube with the Eustachian tube dilation device of claim 1, comprising the steps of:
inspecting a passageway from an ear canal to the Eustachian tube of a subject to determine a geometry of the passageway;
shaping the introducer to provide a shaped introducer having a configuration that substantially matches the determined geometry of the passageway;
inserting the shaped introducer through the passageway to the Eustachian tube;
inserting the balloon catheter through the introducer to place the balloon in the opening of the Eustachian tube; and
inflating the balloon to dilate the Eustachian tube.

16. A method of dilating a Eustachian tube of a subject with the Eustachian tube dilation device of claim 1, comprising the steps of:
inspecting a passageway from an ear canal to the Eustachian tube of a subject to determine a geometry of the passageway;
shaping the introducer to provide a shaped introducer having a configuration substantially matching the determined geometry of the passageway;
inserting the balloon catheter into the shaped introducer;
inserting the shaped introducer with the balloon catheter through the passageway to the Eustachian tube;
positioning the balloon outside the introducer and in the Eustachian tube; and inflating the balloon to dilate the Eustachian tube.

17. The Eustachian tube dilation device of claim 1, further comprising an endoscope integrated with the introducer or the balloon that confirms visually the location of the device in relationship to the carotid artery.

18. The Eustachian tube dilation device of claim 1, wherein the introducer or the balloon further comprises a sensor coil that is wired to an image navigation system that allows the determination of the location of the tip of the introducer or the catheter in relationship to the patient anatomy.
